(19) United States
(12) Patent Application Publication
(10) Pub. No.: US 2025/0025577 A1
(43) Pub. Date: Jan. 23, 2025

(54) TELOMERE EXTENSION AND SUPPORTING REGIMEN FOR PROLONGED HEALTH

(71) Applicant: Erinn Van Wynsberghe, London (CA)

(72) Inventor: Erinn Van Wynsberghe, London (CA)

(21) Appl. No.: 18/714,832

(22) PCT Filed: Dec. 9, 2022
(86) PCT No.: PCT/CA2022/051802

§ 371 Date: May 30, 2024

Related U.S. Application Data
(60) Provisional application No. 63/288,432, filed on Dec. 10, 2021.

(51) Int. Cl. A61K 48/00; C12N 9/22; C12N 15/11; C12N 15/90

(52) U.S. Cl. CPC A61K 48/0058; C12N 9/22; C12N 15/11; C12N 15/907; C12N 2310/20

(57) ABSTRACT

The present invention concerns a method for extending telomeres that have decayed in length without engaging bodily functions such as telomerase, so as to prevent cells from reaching the Hayflick limit and thus avoid risk of becoming senescent. This process is intended to improve the body's ability to restore damaged self-repair mechanisms, cells, and tissues at a pace that is no longer outmatched by senescent cell accumulation, thus reducing probability of age-related maladies including illness, infirmity, disease, and cognitive decline. The present invention also concerns a corresponding regimen of additional procedures and techniques to restore cellular functionality and thus bodily health such as: introducing artificially produced stem cells and/or converting bodily cells to stem cells; doing so in a manner that prevents detection by the body's own immune system; removing senescent cells (“Senolytics”); and replenishing necessary materials extracted during the senolytic process such as P53, all as part of a collective strategy for improving health, health span, and longevity.

### TECHNICAL FIELDS

The present invention relates to the technical field of medical science, specifically the categories of health-span extension and lifespan extension by means of modifying DNA in vitro and in vivo. Technical disciplines involved include:
Cellular biology including the Hayflick limit, and cell death modes including cellular senescence
Genomics including DNA, chromosomes, telomeres, and DNA editing
DNA sampling from a patient—blood, skin, etc.
DNA replication—such as with Polymerase Chain Reaction (PCR) machine
DNA isolation and analysis. DNA editing tools and techniques such as CRISPR-Cas9
Large gene sequence insertion tools such as CAST
Delivery of edited DNA into patients
Artificial stem cell synthesis, such as Induced Pluripotent Stem Cells (IPSCs)
Artificial stem cell delivery into patients
Stem cell modification including universal IPSCs
Modifications to evade the immune system without deploying immunosuppressants
Removing senescent cells from within patients (“Senolytics”)
Replenishing lost materials, enzymes, proteins, etc. (such as P53) due to Senolytics

### DEFINITIONS

Senescence: a condition in which a cell permanently stops dividing but does not die. The senescent cell is not naturally decompiled and recycled within the body but instead emits a range of potentially harmful chemical signals that encourage nearby healthy cells to enter the same senescent state. The continued accumulation of senescent cells is one of the primary causes of aging, bodily decay, and age-related illness, infirmity, neurological decline, disease, and death.

Hayflick Limit: a limit at which normal somatic, differentiated human cells can no longer undergo healthy mitosis and division before risking entering the senescence phase. This limit is reached when the telomeres on the ends of a DNA strand have been completely lost through the replicative process due to the natural end-replication problem.

CRISPR: Clustered Regularly Interspaced Short Palindromic Repeats. It involves a process wherein a DNA strand is spliced and a substitute piece of genetic material is delivered in its place.

Cas9: a CRISPR-associated (Cas) endonuclease (or enzyme) which acts as “molecular scissors” to cut DNA at a location specified by a guide RNA

CAST: CRISPR-Associated Transposons. This process can be used to add an entire gene or large DNA sequence (called a “gene cassette”) to the genome

Guide RNA (gRNA): a type of RNA molecule that binds to Cas9 and specifies, based on the sequence of the gRNA, the location at which Cas9 will cut DNA

PCR: Polymerase Chain Reaction (PCR) machines are cost-effective and highly efficient tools used to amplify segments of DNA or RNA

Senolytics: Selectively inducing death and destruction of senescent cells to improve health in humans

### BACKGROUND INFORMATION

Aging is a phenomenon that detrimentally affects all of humanity.

Aging is characterized by a progressive loss of physiological integrity, leading to impaired function and increased vulnerability to death. This deterioration is the primary risk factor for major human pathologies including cancer, diabetes, cardiovascular disorders, and neurodegenerative diseases (Lopez, 2013).

At the biological level, ageing results from the impact of the accumulation of a wide variety of molecular and cellular damage over time. This leads to a gradual decrease in physical and mental capacity, a growing risk of disease, and ultimately, death (WHO, 2018).

Common conditions in older age include hearing loss, cataracts and refractive errors, back and neck pain and osteoarthritis, chronic obstructive pulmonary disease, diabetes, depression, and dementia. Furthermore, as people age, they are more likely to experience several conditions at the same time (WHO, 2018).

Older age is also characterized by the emergence of several complex health states that tend to occur only later in life and that do not fall into discrete disease categories, commonly called geriatric syndromes. They are often the consequence of multiple underlying factors including frailty, urinary incontinence, falls, delirium and pressure ulcers (WHO, 2018).

Mental health problems are under-identified by health-care professionals and older people themselves, and the stigma surrounding these conditions makes people reluctant to seek help. Over 20% of adults aged 60 and over suffer from a mental or neurological disorder and 6.6% of all disability among people over 60 years is attributed to mental and neurological disorders (WHO, 2017).

The most common mental and neurological disorders in this age group are dementia and depression, which affect approximately 5% and 7% of the world's older population, respectively. Anxiety disorders affect 3.8% of the older population, substance use problems affect almost 1% and around a quarter of deaths from self-harm are among people aged 60 or above. Substance abuse problems among older people are often overlooked or misdiagnosed (WHO, 2017).

Beyond biological changes, ageing is also associated with other life transitions such as retirement, relocation to more appropriate housing, and the death of friends and partners which can further traumatize (WHO, 2018). Older people may experience life stressors such as a significant ongoing loss in capacities and a decline in functional ability, reduced mobility, chronic pain, frailty or other health problems, for which they require some form of long-term care. In addition, older people are more likely to experience events such as bereavement, or a drop in socioeconomic status with retirement. All of these stressors can result in isolation, loneliness or psychological distress in older people, for which they may require long-term care (WHO, 2017).

Additionally, the global population is aging rapidly. Between 2015 and 2050, the proportion of the world's population over 60 years will nearly double, from 12% to 22% (WHO, 2017). Such a sharp increase in elderly persons is likely to incur significant economic strain globally in providing care and support services for the elderly and infirm.

Further, many people suffer from diseases which accelerate the aging process and its corresponding ailments, despite the patient being comparatively young or even in childhood. For example, “telomere syndromes” are inherited conditions that can cause numerous symptoms including bone marrow failure, lung disease, pulmonary fibrosis (scarring of the lung), liver disease (cirrhosis), gastrointestinal disease, insufficient weight gain and physical growth, skin and mucosal abnormalities, abnormal skin pigmentation, overabundance of spots and patches, nail weakness and hair loss, eye problems, skeletal problems (ie. osteoporosis), narrowing of the esophagus, and cancer risk (Potter, 2017).

For these reasons, aging is a major concern worldwide.

Finding ways to reduce such age-related suffering would lead to numerous benefits. People could live longer and healthier lives, avoiding age-related illnesses and diseases. Health care costs for senior citizens could be drastically lowered, thus reducing economic hardship for all peoples everywhere and for governments who manage health care services. And, genuine lifespan extension could bring about substantial improvement for all of humanity including hope of elevating out of poverty and finding new opportunities for inclusion, participation, contribution, and achievement.

As of this writing, there is some scientifically-grounded understanding of the causes of aging. Generally, the time-dependent accumulation of cellular damage is widely considered the cause of aging (Lopez-Otin, 2013). One system of classification is the “hallmarks of aging” which include: genomic instability, telomere attrition, epigenetic alterations, loss of proteostasis, deregulated nutrient-sensing, mitochondrial dysfunction, cellular senescence, stem cell exhaustion, and altered intercellular communication (Lopez-Otin, 2013).

Of this list, “telomere attrition” is of particular importance because it directly leads to the other conditions listed. Telomeres are in essence the protective end-caps of DNA, preserving the integrity of the genome. They consist of specialized non-coding DNA repeat sequences (repeats of “TTAGGG”). Mammalian telomeres might have 1-50 kb of telomeric DNA, which becomes 40-200 base pairs shorter after each cell division cycle, and becomes 5-8 kilobase shorter during senescence (Erdem, 2021).

Despite the inherent problem of telomere attrition during cell division (mitosis), the process of cell division is fundamentally essential for organism growth, reproduction, and tissue repair: Organisms grow either by augmenting cell size or increasing in cell number; from conception, cells divide at accelerated rates to increase the size of the organism, and cells continue to divide to increase organism size until the organism reaches adulthood; and, when injury occurs, cells divide to replace old, dead, or damaged cells. At each of these divisions, telomeres are reduced in length.

When telomeres become too short, cells can no longer divide and the cell enters a new phase of existence. Various outcomes are possible including apoptosis, a form of programmed cell death in which the cell systematically disassembles itself and the constituent parts are recycled within the body and re-purposed. Conversely, a second form of cell death, necrosis, destroy the defunct cells without strategy, often as a response to an overwhelming stress such as a traumatic injury or exposure to poison. Cells swell up and burst, causing the afflicted body region to become inflamed and sensitive (Dutchen, 2011).

However, there is a third outcome for telomere attrition called senescence, in which a cell stops dividing but does not die. Over time, large numbers of old (or senescent) cells can build up in tissues throughout the body, releasing harmful substances that may cause inflammation and damage to nearby healthy cells. Senescence thus may play a role in the development of cancer and other diseases (NCI. b, n.d.).

Additional impacts of accumulated senescent cells:
Interrupted inter-cellular communication: Altered communication among cells is mainly caused by hallmarks such as cellular senescence (IMYu, 2021). Senescent cells are known to secrete an inflammatory, immunosuppressive, and harmful mixture known as senescence-associated secretory phenotype (SASP) which inflames neighboring cells. When inflammation activates NF-KB in the hypothalamus, it inhibits the production of GnRH. This hormone is used to signal other bodily systems, and its reduction may contribute to bone fragility, muscle weakness, skin degradation, and other harmful effects with age (IMYu, 2021). Further, senescent cells communicate with neighboring cells by direct intercellular protein transfer (IPT). Transferred proteins such as natural killer (NK) cells lead to increased cytotoxicity and might impact tumorigenesis and tissue aging (Biran, 2015).
Impeded mitochondrial function: The enzyme nicotinamide adenine dinucleotide (NAD+) along with many other molecules responsible for maintaining cell functionality, maintenance, and self-repair typically decline with age. Their populations within the body are directly affected by the presence of senescent cells. Specifically, inflammatory cytokines secreted by senescent cells induce macrophages to proliferate and express high levels of CD38 which consumes NAD+ (Covarrubias, 2020). Further, Senescent CD8+ T cells exhibit mitochondrial dysfunction, increased production of reactive oxygen species (ROS), and impaired mitochondrial biogenesis (Akbar, 2016).
Loss of proteostasis: Senescence has been recently shown to directly lead to proteostasis collapse in human cells. Proteostasis is the regulation of a balanced, functional proteome (the complete set of proteins expressed by an organism). Senescent cells possess a deteriorated ability to regulate stress-related transcriptional programs and as such proteostasis is disrupted in a cell autonomous manner (Montague-Cardoso, 2021;Sabath, 2020).
Epigenetic alterations: Senescent cells undergo nuclear phenotypic changes wherein the epigenetic and chromatin landscape undergo widespread alterations (Nacarelli, 2017). As of this writing, direct links in humans are still to be fully clarified, but animal testing has provided insights. For example, tests in mid-life flies show enhanced acetyl-CoA, increased histone acetylation (change in charge of histone tail), and longevity-altering transcriptome changes (Yang, 2018).
Deregulated nutrient-sensing: Reduced responsiveness of nutrient sensing pathways is widely associated with human disease and ageing. Cellular senescence causes mTORC1 to become resistant to nutrient starvation, leaving the kinase complex active which has been associated with defects in both growth factor and amino acid sensing pathways. The observed insensitivity of mTORC1 to changes in the balance of mitogenic cues is likely to have wide implications for protein translation and cell metabolism (Carroll, 2018). Further, as discussed above, senescent cells promote NAD+ decline via the activation of CD38+ macrophages (Covarrubias, 2020). NAD+ is a critical component in maintaining metabolism and catabolismby producing and regulating Sirtuins which in turn regulate cellular health (Bajnath, 2021).
Stem cell exhaustion: Aging induces cellular and molecular changes including modification of stem cell pools. In particular, alterations in aging neural stem cells (NSCs) are linked to age-related cognitive decline (de Lucia, 2020). As of this writing, senescent cells are suspected of exhausting tissue of proliferation-competent cells and renewable stem cells over time, diminishing homeostasis and regenerative capacity of the tissue (Nacarelli, 2017).
Genomic instability: Increased tendency of genome alteration during cell division can lead to cancer. It is caused by defects in certain processes that control the way cells divide including: mutations in certain genes involved in repairing damaged DNA; mistakes that don't get corrected when DNA is copied in a cell; and defects such as broken, missing, rearranged, or extra chromosomes (NCI. a, n.d.). While those are the most common causes, genome instability can be enhanced by exposure to external genotoxic agents or as the result of cellular pathologies (Aguilera, 2013).

Genomic instability is therefore one exception to senescence, because one of its major contributors (in addition to telomere length homeostasis) is maintenance of ribosomal DNA (rDNA) stability (Lee, 2021).

Thus, telomere attrition is one direct cause of senescence, and subsequently a catalyst for most of the aforementioned hallmarks of aging. Developing a means of preventing telomere attrition or re-establishing telomere length would therefore be of paramount value in preventing and/or remediating age-related problems.

One method of preventing cellular senescence would be to ensure that all dying cells terminate by a controlled means such as programmed cell death (PDC) including apoptosis, autolysis, pyroptosis and others, or by necrotic cell death including necrosis, rather than entering a state of permanent cell cycle arrest characterised by senescence.

However, even if such a procedure were possible, this would still result in the natural death of all cells and so lead to decay and its related illnesses for the patient, since all telomeres in all cells would still eventually decay to zero length and thus all cells would eventually die, preventing any further mitosis and resulting in age-related illness.

An alternative means of preventing cellular senescence would be to prevent the cells from reaching the Hayflick limit at all, and so avoiding cell death and senescence altogether. This would also provide the additional bonus of allowing the organism to continue living and staying healthy and robust for much longer than the natural lifespan, wherein cells could continue dividing as if they had been reverted back to a younger biological age. One means of achieving this would be to find a way to extend telomeres. Much work has been done on this proposed process, but have inherent problems.

### LIMITATIONS OF PRIOR ART

Numerous organizations, firms, groups, and individuals have advocated lifestyle changes as a means of reducing the pace of telomere attrition. Techniques have included changes to regular diet and intermittent fasting to achieve and maintain healthy weight, increased amount of exercise, improving sleep habits, reducing and managing chronic stress, eating a telomere-protective diet, and incorporating supplements.

However, this option presents numerous burdens and drawbacks:
Significant lifestyle change requirement
Constant and continual vigilance and expenditure
No guarantee of success
No formal metrics nor effective means to monitor progress or failure
Most importantly, this process only SLOWS telomere depletion. It does extend telomeres, and so only delays the inevitable telomere reduction, cellular senescence, aging, and age-related impairments

Other inventors have faced problems in this area of re-extending telomeres to promote longer and healthier lives. Some groups have attempted to extend telomeres by direct intervention at the molecular level. Specifically, some have delivered a modified RNA that encodes a telomere-extending protein to cultured human cells. Cell proliferation capacity was dramatically increased, yielding large numbers of cells for study. Skin cells with telomeres lengthened by the procedure were able to divide up to 40 more times than untreated cells, lengthening human telomeres by as much as 1,000 nucleotides—the equivalent of many years of human life (Stanford New Center, 2015). Three successive transfections over a 4-day period produced extended telomeres of up to 0.9 kb (Ramunas, 2015).

The RNA used contained the coding sequence for TERT, the active component of a naturally occurring enzyme called telomerase. Telomerase is expressed by stem cells, including those that give rise to sperm and egg cells, to ensure that the telomeres of these cells stay in ideal condition for the next generation. Most other types of cells, however, express very low levels of telomerase (Stanford New Center, 2015).

Methods such as this, although demonstrably effective, could have some specific limitations:
The RNA delivered is dependent on engagement with host stems cells, which in an older person are usually in much shorter supply, limiting potential efficacy
The length to which telomeres are extended is dependent on the degree of repetition of the process, such that if a significant alteration is desired (such as more than a decade's worth of age reduction), then a significantly high number of injections might be required, adding further risk for complications, failure, or infection
The method forces participation of various bodily components within the cell environment including telomerase, which could have further unforeseen consequences including but not limited to the incidental support of the growth of cancerous cells
The method has no means of distinguishing between healthy vs damaged, tumorous, or cancerous DNA, and thus no ability to selectively operate. It could promote growth of cancerous cells or damaged DNA.

### NOVELTY OF PROPOSED SOLUTION

The present invention will improve upon the prior art by directly resolving their shortcomings (discussed above) to achieve the following:
As much telomere growth as desired with a single application of the present invention, or short series of applications of such (reducing risk of potential harm, lowering burden on the patient)
Specific and precise telomere length can be achieved to suit preference
No manipulation of bodily functions (won't force engagement of telomerase)
No dependence on other cellular functions such as telomerase of stem cells
No impact on other bodily elements such as amount of telomerase in the system
Won't have to be repeated often (perhaps once a decade or generation)
No limit to benefits (can chose any desired telomere length)
Won't impact cells with cancerous or tumorous DNA at all
   (won't help them, won't hurt them, simply won't engage with them at all)
Could be undone/reversed if desired (markers are installed to allow for undoing)
No lifestyle change imposed
No need for constant and continual vigilance and expenditure
Precise metrics for success (follow-up tests with telomere measurements)
Can apply to all patients

Novel use of CRISPR: The present invention changes the nature of the use of gene editing from replacing select genes to instead affecting the anticipated life of the cell, preventing it from reaching the Hayflick limit and so preventing decay to senescence.

Novel search function: The present invention is additionally novel in that it introduces a new screening methodology, that of carrying the entire DNA strand as its search function so that it can find and subsequently perform edits on only the cells which completely satisfy the intended criteria, avoiding risk of editing (and extending the life of) cancerous, damaged, or senescent cells.

Novel location of splice: CRISPR (and related gene editing applications) is traditionally applied throughout the body proper of the DNA strand, whereas the present invention proposes a novel use by targeting the specific junction where the end of the DNA strand proper meets the beginning telomere,

Novel size of replacement: The present invention is further novel in that it does not replace a small segment with another, but rather replaces one bulk section with another, capitalizing on the most recent tools in CAST.

Replace rather than insert: The present invention is also novel because rather than an insertion, it provides a replacement. The tail of the DNA strand is replaced with a new tail, and it should be or no consequence if the severed piece (telomere with some of the DNA strand) is reconnected or not. If that severed piece is lost, then the DNA will still be fully functional.

Novel two-step process: The present invention involves two distinct engagement points with the patient (DNA sampling and solution injection) which provides greater protection against accidental, inappropriate, or weaponized use.

Easy adaptability: The present invention is novel in that it is not specific to any one organism. Significant tailoring will not be required in order to change from using it on one creature to another, other than taking a new sample for the patient. Thus, it can be applied to any life form with telomeres that reduce in length during cell division. Because of this, lab tests can be conducted in organisms of escalating complexity in rapid succession, such as from (male) mosquitos and other short-lived insects, to mice and other small mammals, to apes and chimps, and finally to humans. Such convenience will greatly reduce time, cost, complexity, and risk in the development, testing, and validation of therapeutics for commercial use.

### LIST OF DRAWINGS

FIG. 1. One example iteration of the genome editing process with CRISPR-Cas9

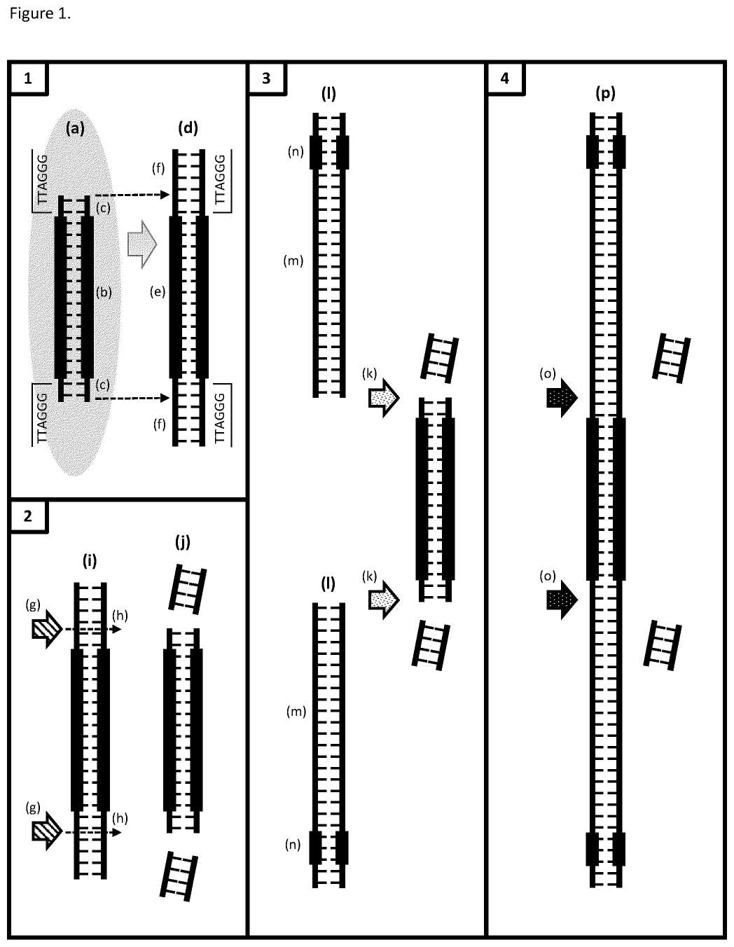

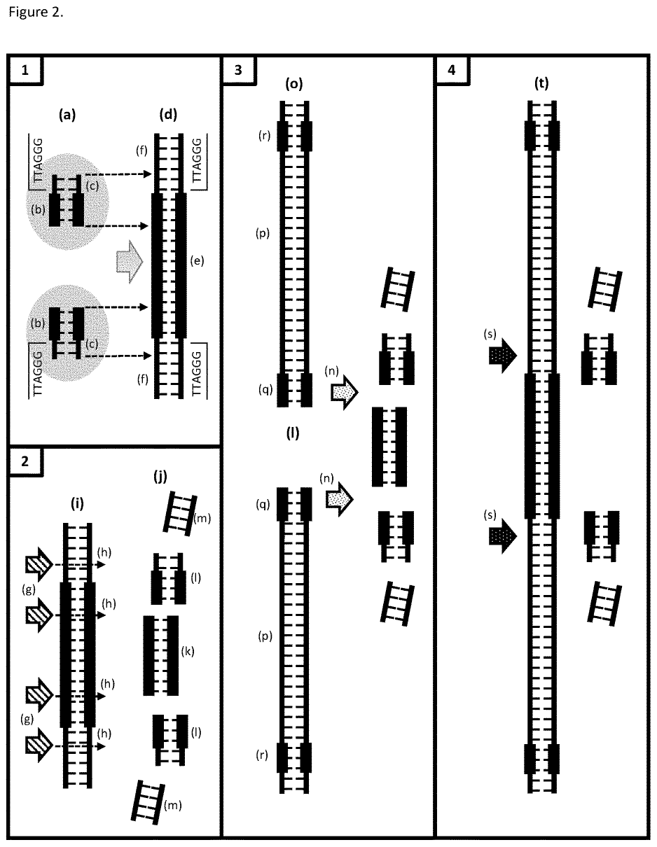

Not drawn to scale. DNA strands and gene editing tools visualized as vastly shorter and far less complex, to focus attention on the critical elements of the procedure.

### Frame [1]

(a) the Guide RNA (gRNA) comprised of (b) the entire target DNA strand proper, plus (c) some portion of telomere sequences (labelled as TTAGGG); (grey arrow) seeks out and finds a match (dotted lines), and aligns with (d) the DNA in the patient body comprised of (e) the DNA body proper and (f) both telomere strands.

### Frame [2]

(g) the Cas9 (or other Cas option or other DNA cutting implement) (striped arrow) performs (dotted line) a double strand break at (h) a single incision point, at each end of (i) the patient's DNA proper, just past the beginning of the telomeres. Column (j) visualizes the separation gap now formed between the original DNA sequence proper and the now-severed end piece.

### Frame [3]

(k)the gene editor deploys (speckled arrow) (I) the replacement DNA strand consisting of (m) the elongated telomere sequence and (n) a near-end marker

### Frame [4]

(o) the body's natural DNA repair process (dark spotted arrow) rejoins the DNA strand with the replacement piece to form (p) a new solid DNA strand.

This is one of numerous ways that the genome edit of the present invention can be deployed.

FIG. 2. Another example iteration of the genome editing process with CRISPR-Cas 9

Not drawn to scale. DNA strands and gene editing tools visualized as vastly shorter and far less complex, to focus attention on the critical elements of the procedure.

### Frame [1]

(a) the Guide RNA (gRNA) comprised of (b) a portion of the DNA strand sampled from the patient where the last of the DNA strand proper naturally met the telomeres, plus (c) some limited portion of telomere sequences (labelled as TTAGGG); (grey arrow) seeks out and finds a match (dotted lines) with (d) the DNA in the patient's body, aligning where it matches between (e) the DNA body proper and (f) the telomere strands, at both ends of the patient's DNA.

### Frame [2]

(g) the Cas9 (or other Cas option or other DNA cutting implement) (striped arrow) performs (dotted line) a double strand break at (h) four incision points-each in the vicinity of where the DNA strand proper meets the telomeres, at both ends of (i) the patient's DNA. Column (j) visualizes the separation gaps now formed between (k) the original DNA sequence proper plus (I) the now-severed pieces and (m) the original telomere ends (of undetermined length).

### Frame [3]

(n) the gene editor (speckled arrow) deploys (o) the replacement DNA strand consisting of (p) the elongated telomere sequence with (q) a replacement for the portion of DNA strand proper that was excised in the splice, and (r) a near-end marker.

### Frame [4]

(s) the body's natural DNA repair process (dark spotted arrow) rejoins the DNA strand with the replacement piece to form (t) a new solid DNA strand.

This is also one of numerous ways that the genome edit of the present invention can be deployed.

### DETAILED DESCRIPTION

The present invention concerns a gene editing process by which a patient is given a gene-edited material (by injection, ingestion, or any other acceptable delivery method), sufficient in quantity and quality to modify all desired chromosomes and achieve extension of all telomeres to desired length. Within the present invention the patient will also be provided with additional materials which will assist with reduction of conditions that lead to age-related maladies (including but not limited to artificially produced stem cell supplements; senolytic products to remove senescent cells; methods to replenish reduced materials of need including enzymes and proteins; and methods to ensure that processes are successful without interference from the body's natural immune system).

To produce the genome-editing material of the present invention, one should undertake the following three critical steps:
(i) Prepare a sample material to search for inside the patient (“guide RNA”)
(ii) Prepare a replacement material to insert into the patient's DNA strand
(iii) Select a cutting position within the patient's DNA strand

Note that any genome editing tool may be chosen such as CRISPR and its family of processes, TALEN, ZFN, LEAPER, PRIME, or any not mentioned or yet to be invented. The present invention can be achieved invention as long as the gene editing tool selected can perform (i) a precise detection within a chromosome based on the carried guide RNA sample, (ii) a precise double break DNA splice, and (iii) introduce a carried piece of DNA for integration and subsequent DNA re-suturing.

Details on steps to produce the present invention are as follows:
(i) Genome Edit: The “search” material is prepared as follows:

### Obtain DNA Sample from Patient

Sources can include but are not limited to blood, skin, hair, buccal (cheek) swabs, saliva, hair, nails, blood, or sperm. Procedures and tools required will be familiar to someone skilled in the art of obtaining such samples.

### Extract and Isolate DNA from Sample

Options include but are not limited to Phenol-chloroform isoamyl alcohol, Proteinase K, CTAB method, spin column-based methods and magnetic bead-based technique. Method of choice will depend on the sample type and Purity and yield of DNA obtained. Procedures and tools required will be familiar to someone skilled in the art of isolating DNA.

### Replicate Sample

Create millions or billions of copies in rapid fashion. Utilize methods including but not limited to Polymerase chain reaction (PCR). Procedures and tools required will be familiar to someone skilled in the art of DNA replication.

### Identify and Isolate Target DNA

Examine DNA samples to identify which is the best example of a health strand. Remove and damaged or otherwise undesirable DNA samples. Procedures and tools required will be familiar to someone skilled in the art of analyzing and separating DNA.

### Replicate Target Sample

Create numerous copies of the DNA in rapid fashion. Utilize methods including but not limited to Polymerase chain reaction (PCR). Procedures and tools required will be familiar to someone skilled in the art of DNA replication and the above process of replicating the original sample.

### Prepare Search Material (Guide RNA)

Prepare the above specialized sample to serve as the guide Ribonucleic acid (RNA) in the gene editing process.

The guide RNA should also include a distinct number of telomere sequences at the end of the DNA strand to ensure that only DNA of healthy cells are targeted which still have several mitosis sessions left. Thus, the target cells will have not yet reached nor will be in eminent danger of reaching, the Hayflick limit and so not about to undergo cellular senescence, apoptosis, or other possible forms of cell death or damage. Procedures and tools required will be familiar to someone skilled in the art of preparing genome editing processes such as CRISPR (or any of the many listed previously, or yet to be developed).

The reason that the present invention involves searching the entire DNA strand is to avoid the risk of increasing telomere length on a chromosome that is faulty, mutated, or cancerous, thus avoiding increasing the lifespan of such a cell. The present invention could be undertaken in such as way as to only search a portion of the DNA adjacent to the beginning of the telomere, which might make it easier for replicating that material and assuring more success in the process, but it is recommended not to pursue that latter course of action.
(ii) Genome Edit (ii): The “replacement” material is created as follows:

### Prepare Telomere Chain

The replacement material should be an extremely long chain of telomeres, of such length as would normally be found in a human body at peak lifespan with maximal cell division capability remaining (ideally 8,000-10,000 nucleotides long). This material can be prepared by modifying a sample from a patient, or by producing it artificially in a lab. Synthetic synthesis of DNA does not require template DNA, which should allow virtually any DNA sequence to be synthesized in the laboratory.

Such a process can be undertaken in two key steps:
(1) Solid-phase DNA synthesis (aka DNA printing) to produce oligonucleotide fragments that are generally under 200 base pairs
(2) Connect the oligonucleotide fragments using various DNA assembly methods

To ensure that significantly long strands of telomere can be carried by the gene editor (ie. CRISPR) and delivered to the chromosome, the present invention includes the use of tools and techniques such as CRISPR-associated transposons (CASTs). This tool can mediate highly efficient, RNA-guided insertion of an entire gene or large DNA sequence (cargo DNA) to the genome, such as the extensively long telomeres of the present invention. Instructions on applying the CAST method are provided by Rybarski et. al (2021) and Zhang et. al (2020) among others. Details on the diverse number of CAST options available are provided by Rybarski et. al (2021).

### Prepare and Append Biomarker

Further, the present invention also introduces the option of including a customized identifying marker within the replacement sequence to help identify the efficacy of the procedure at a later date. One of many possible methods to implant this marker would be to have the first (and last) of the introduced telomere sequences to be a reverse or jumbling of the normal telomere order.

Such an option would not occur in nature and so would allow future detection of the success of the operation over a longer period of time, and if necessary, allow for the targeting of additional editing in only cells that underwent the previous process.

Procedures and tools required will be familiar to someone skilled in the art of synthetic biology, namely constructing and assembling genes from nucleotides.
(iii) Genome Edit (iii): The “splice point” is chosen and prepared as follows:

Select a position outside of the DNA proper, and into the telomeres to avoid any risk of damage to the valuable DNA

Select a position ideally far enough into the telomeres that there will be no risk of damage to the DNA proper, and no risk that the cell might currently be in the process of mitosis, or cell death (senescence, apoptosis, etc.). Although such ignored cells will continue to decay and will soon reach life cycle termination and so risk entering senescence, it is advisable that such cells be ignored for this procedure so as to prevent risk of creating long-living cells with fundamentally damaged chromosomes or with cellular disfunctions.

Ideally, select a cutting position BEFORE the final detection point, such as the length of one telomere sequence.

Procedures and tools required will be familiar to someone skilled in the art of precision genome editing, involving processes such as CRISPR.

### Deliver to Patient

After the genome-editing material is prepared, it should be delivered to the patient as a single or series of injections, or other appropriate delivery method. The patient should then be monitored overall an extended period of time, possibly up to at least one year to account for the slowest natural mitosis rates in the body to confirm efficacy. Procedures, processes, and tools involved should all be familiar to someone skilled in the art of medicine administration, specifically gene editing therapeutics.

### Additional Telomere Delivery Method

If a CRISPR process can only insert a certain length of telomere (gene cassette) shorter than the final desired telomere length, then the present invention can still be achieved by dividing the work into stages across several treatments, with each CRISPR's guide RNA searching for the end of the last addition, then adding another portion of telomere to that end piece.

This can be accomplished in several ways. One possible option would be for the first CRISPR-Cas9 sent in to have within its replacement sequence an identifying marker which is unique and differentiated (from both telomeres and normal DNA sequences), such as a reverse of the standard telomere sequence, or a partial segment of the telomere sequence TTAGGG which will not cause complications for normal RNA reading and bodily operations. Thus, the next incoming CRISPR will detect that marker and be positioned at its location for appending. The new CRISPR will perform the DNA splice, removing the old marker before laying down its segment of telomere plus a corresponding new marker at the end. Therefore, successive CRISPR treatments will each find the target marker, then remove it before adding a telomere extension with new marker, and so on, until DNA tests show that average telomere length has reached what was intended.

Further, if desired, the identifying marker of each successive CRISPR regimen can be unique in and of itself, to ensure that only targeted DNA strands are selected for further editing (thus avoiding any cells not desired for the procedure).

### Alternative Method to Extend Telomeres at End Points

An alternative iteration of the present invention is to prepare a gene editor which seeks out the end of a telomere within the DNA of the patient, rather than targeting the location where the strand proper meets the beginning of the telomeres.

In such a situation, the Guide RNA would contain a segment of telomere, along with information or instructions for it to seek out where the telomere ends. The gene editor (ie. CRISPR) delivers a replacement strand consisting of a long sequence of telomeres plus an optional end market or near-end marker. The gene editor would perform at splice (if desired) at or near the end of the patient DNA, and the replacement strand would be deposited, allowing the body's natural processes to suture in the change.

This alternative embodiment could be applied in addition to, or in place of, the aforementioned methods.

### Additional Regimen—Evade the Immune System without Employing Immunosuppression

The processes described above and herein may be inefficient or vulnerable to failure because the body's natural immune system might fight against the new materials and reject the procedure, preventing affective treatment.

To avoid such an outcome, the present invention also includes a method of gene editing which can make inclusions and additions which are undetectable to the immune system, thus making it possible to carry out the cell therapy transplants of the present invention without having to take the additional and risky steps of artificially supressing the patient's immune response.

Hypo-immune stem cells can be created which evade the immune system, and can be used to generate cells of a desired type that can be transplanted into any patient without the need for immune-suppression, since the cells won't illicit an immune response. CRISPR-Cas9 (or a similar gene editing tool) can be used to remove genes involved in the major histocompatibility complex to appear less foreign or invasive, and simultaneously increase expression of a protein that emits a signal to protect cells from macrophages. This process will increase the probability of success of the delivery of new genetic material into the patient. The specific steps and methodology for preparing cells to be less detectable by the immune system is described by Deuse et. al (2021).

### Additional Regimen for Cellular Rejuvenation

The processes described above will extend telomeres to dramatically reduce the rate of new senescent cell production, thus affording the body with greater time to engage natural processes of removing existing senescent cells and so make meaningful strides to restoring the body to a state of greater physiological integrity.

However, a body that has already reached a significant degree of age-related decay and decline may need additional assistance to expedite bodily repairs including assistance with addressing accumulated senescent cells, depleted stem cells, and reduced functionality of repair mechanisms such as NAD+. To assist the patient in this regard, the present invention includes the following additional measures:

Introduce Stem Cells: Providing the body with additional stem cells can accelerate repair and healing. Stem cells can be artificially produced (such as with induced pluripotent stem cells) for convenience and reduced cost. Further, universal IPSCs can be delivered from any donor to any patient regardless of blood type or other mitigating factors to reduce time and cost in the procedure. In one possible iteration of the present invention, IPSCs can be more effectively delivered if they exclude certain Yamanaka factors (epigenome proteins removed from sample cells to convert them into pluripotent stem cells).

Prevent Autoimmune Engagement: Modify the materials to be delivered into the patient (such as IPSCs) to reduce or eliminate the prospect of triggering the body's immune response, improving the probability of process success. Processes and procedures for reducing detectability are described by Deuse et. al (2021).

Remove Senescent Cells: Processes such as Senolytics can remove senescent cells from the body by decomposing them, further aiding the body in restoring health and functionality

Replenish Lost Materials: Some vital bodily materials can become inadvertently depleted during the senolytic process such as the enzyme P53. It will be advantageous to resupply the patient with such assets. Testing of the patient will be required to determine how much of each substance was lost, and the appropriate method to replenish based on established medical procedures.

### Uses

There are many possible uses for the present invention. Benefits to the patient could include (but are not limited to):
Restore physiological integrity
Increase the speed and efficiency for the body to replace damaged cells, to repair and heal
Reduce probability and severity of age-related maladies including infirmity, disease, and cognitive decline
Reduce or reverse common conditions of older age include hearing loss, cataracts and refractive errors, diminished vision, back and neck pain, osteoarthritis, chronic obstructive pulmonary disease, diabetes, dementia, frailty, urinary incontinence, delirium, pressure ulcers, and mental and neurological disorders
Reduce derivative health complications from old age such as depression, substance abuse, falls, bodily pain, self harm, or elder abuse
Reduce risk of major human pathologies including cancer, diabetes, cardiovascular disorders, and neurodegenerative diseases
Reduce or remove the damages caused by aging such as wrinkles, liver spots, calcium deposits, brittle bones, etc.
Extend health span (amount of time lived in a healthy condition)
Extend lifespan
Increase ability to live active, productive lives
Overcome diseases wherein telomeres diminish prematurely such as telomere syndromes, reducing risk or alleviating conditions including bone marrow failure, lung disease, pulmonary fibrosis (scarring of the lung), liver disease (cirrhosis), gastrointestinal disease, insufficient weight gain and physical growth, skin and mucosal abnormalities, abnormal skin pigmentation, overabundance of spots and patches, nail weakness and hair loss, eye problems, skeletal problems (ie. osteoporosis), narrowing of the esophagus

### Example Application

A detailed example of how to apply the present invention is presented. This is one of many possible approaches, which could be altered or adapted by one who is skilled in the art of therapeutic delivery.
An organization advertises the service described in the present invention
An interested customer may wish to reduce their biological age, or may be suffering from a condition that prematurely deteriorates their telomere length
The interested customer contacts the organization and books an appointment, becoming a patient
The patient undergoes an education process to ensure meaningful consent
The patient commits to the service
The organization extracts a DNA sample from the patient
The organization replicates the DNA sample (such as with PCR machine)
The organization prepares a CRISPR wherein the search function is the patient's entire DNA strand (from the sample provided above) plus several telomeres (to assure ignoring of senescent cells). The target splice position is near the end of the search function. The replacement strand includes a custom signature marker (such as a telomere backwards or jumbled) followed by an extremely long strand of telomere such that the patient's DNA after the procedure would be indistinguishable from that of a young person (post-adolescence).
The organization delivers the CRIPSR to the patient either in one dose or a series of repeated doses across a period of time, and monitors their health.
The patient returns to the organization for testing and monitoring across the span of an extended period of time, perhaps as long as one year, to ensure process efficacy and continued good health

This is one of many possible approaches, and could be altered, adapted, or substituted by one who is skilled in the art of therapeutics delivery and medical facility administration.

### Advisories

As of this writing, human-based in vivo genome editing procedures such as with CRISPR and the others listed above are relatively novel and not widespread. Therefore, standard operating procedures (SOP) in a facility attempting to perform the present invention might not inherently include all necessary safety precautions. Further, the present invention involves a novel process that will affect the entire body and do so over the course of the entire cellular regeneration cycle, which could last as long as one year for each cell to undergo mitosis and thus show efficacy under testing. Therefore, some additional processes are recommended to ensure patient safety and well-being which could include but are not limited to:
Ensure sufficient understanding for informed patient consent
Ensure legality and regulatory approval in region from local to national levels and investigate for additional requirements unique to this procedure.
Test patient immediately afterward to ensure efficacy and well-being
Monitor and test patient regularly over a long duration to ensure progress and health. The total process might take a full calendar year to complete and should be monitored throughout
Have measures in place to terminate and/or undo the procedure as best as possible in the event of complications or problems

Further, not all genome-editing tools may be viable candidates if they cannot carry a sufficiently long DNA strand as the guide RNA, and cannot carry the telomere of desired length as the replacement DNA. One adaptation of the present invention is that the guide RNA could be only a portion of the overall DNA, precise to a much shorter interval to ensure successful locating of the desired cutting position. However, this would risk the possibility that mutated or cancerous chromosomes become selected for the proposed process of telomere extension—an undesirable outcome.

### REFERENCES

Aguilera, A., García-Muse, T. (2013). Causes of Genome Instability. Annual Review of Genetics, 47(1), 1-32. https://doi.org/10.1146/annurev-genet-111212-133232
Akbar, A. N. (2016). The convergence of senescence and nutrient sensing during lymphocyte ageing. Clinical & Experimental Immunology, 187(1), 4-5. https://doi.org/10.1111/cei.12876
Bajnath, A. (2021 March 22). DEREGULATED NUTRIENT-SENSING—A Fine Line Between Longevity and Aging. The Institute for Human Optimization. https://ifho.org/deregulated-nutrient-sensing-a-fine-line-between-longevity-and-aging/.
Biran, A., Perelmutter, M., Gal, H., Burton, D. G. A., Ovadya, Y., Vadai, E., Geiger, T., Krizhanovsky, V. (2015). Senescent cells communicate via intercellular protein transfer. Genes & Development, 29(8), 791-802. https://doi.org/10.1101/gad.259341.115
Carroll, B., & Korolchuk, V. I. (2018). Nutrient sensing, growth and senescence. The FEBS Journal, 285(11), 1948-1958. https://doi.org/10.1111/febs.1440
Covarrubias, A. J., Kale, A., Perrone, R., Lopez-Dominguez, J. A., Pisco, A. O., Kasler, H. G., Schmidt, M. S., Heckenbach, I., Kwok, R., Wiley, C. D., Wong, H.-S., Gibbs, E., Iyer, S. S., Basisty, N., Wu, Q., Kim, I.-J., Silva, E., Vitangcol, K., Shin, K.-O., . . . Verdin, E. (2020). Senescent cells promote tissue NAD+ decline during ageing via the activation of CD38+ macrophages. Nature Metabolism, 2(11), 1265-1283. https://doi.org/10.1038/s42255-020-00305-3
de Lucia, C., Murphy, T., Steves, C. J., Dobson, R. J., Proitsi, P., Thuret, S. (2020). Lifestyle mediates the role of nutrient-sensing pathways in cognitive aging: cellular and epidemiological evidence. Communications Biology, 3(1). https://doi.org/10.1038/s42003-020-0844-1
Deuse, T., Tediashvili, G., Hu, X., Gravina, A., Tamenang, A., Wang, D., Connolly, A., Mueller, C., Mallavia, B., Looney, M. R., Alawi, M., Lanier, L. L., Schrepfer, S. (2021). Hypoimmune induced pluripotent stem cell-derived cell therapeutics treat cardiovascular and pulmonary diseases in immunocompetent allogeneic mice. Proceedings of the National Academy of Sciences, 118 (28). https://doi.org/10.1073/pnas.202209111
Dutchen, S. (2011 February 23). Cell Suicide: An Essential Part of Life. LiveScience. https://www.livescience.com/12949-cell-suicide-apoptosis-nih.html.
Erdem, H. B., Bahsi, T., Ergun, M. A. (2021). Function of telomere in aging and age related diseases. Environmental Toxicology and Pharmacology, 85 103641. https://doi.org/10.1016/j.etap.2021.103641
IMYu, Research, U. C. B., Lifespan Extension Advocacy Foundation. (2021 May 13). Why we Age: Altered Intercellular Communication. Lifespanio. https://www.lifespan.io/topic/altered-intercellular-communication/.
Lee, J. W., Ong, E. B. (2021). Genomic Instability and Cellular Senescence: Lessons from the Budding Yeast. Frontiers in Cell and Developmental Biology, 8. https://doi.org/10.3389/fcell.2020.61912
Lopez-Otin, C., Blasco, M. A., Partridge, L., Serrano, M., Kroemer, G. (2013). The Hallmarks of Aging. Cell, 153(6), 1194-1217. https://doi.org/10.1016/j.cell.2013.05.039
Montague-Cardoso, K. (2021). Cellular proteostasis decline in human senescence. Communications Biology, 4(1). https://doi.org/10.1038/s42003-020-01578-w
Nacarelli, T., Liu, P., Zhang, R. (2017). Epigenetic Basis of Cellular Senescence and Its Implications in Aging. Genes, 8(12), 343. https://doi.org/10.3390/genes8120343
NCI. a (n.d.). NCI Dictionary of Cancer Terms. National Cancer Institute. https://www.cancer.gov/publications/dictionaries/cancer-terms/def/genomic-instability.
NCI. b (n.d.). NCI Dictionary of Cancer Terms. National Cancer Institute. https://www.cancer.gov/publications/dictionaries/cancer-terms/def/senescence.
Potter, M. (2017 July 18). Telomere Syndromes and Dyskeratosis Congenita. Johns Hopkins Kimmel Cancer Center.
https://www.hopkinsmedicine.org/kimmel_cancer_center/cancers_we_treat/b one_marrow_failure_disorders/telomere_syndromes.html.
Ramunas, J., Yakubov, E., Brady, J. J., Corbel, S. Y., Holbrook, C., Brandt, M., Stein, J., Santiago, J. G., Cooke, J. P., Blau, H. M. (2015). Transient delivery of modified mRNA encoding TERT rapidly extends telomeres in human cells. The FASEB Journal, 29(5), 1930-1939. https://doi.org/10.1096/fj. 14-259531
Rybarski, J. R., Hu, K., Hill, A. M., Wilke, C. O., Finkelstein, I. J. (2021). Metagenomic discovery of CRISPR-associated transposons. Proceedings of the National Academy of Sciences, 118(49). https://doi.org/10.1073/pnas.2112279118
Sabath, N., Levy-Adam, F., Younis, A., Rozales, K., Meller, A., Hadar, S., Soueid-Baumgarten, S., Shalgi, R. (2020). Cellular proteostasis decline in human senescence. Proceedings of the National Academy of Sciences, 117(50), 31902-31913. https://doi.org/10.1073/pnas.2018138117
Stanford News Center. (2015 January 22). Telomere extension turns back aging clock in cultured human cells, study finds. Stanford News Center. https://med.stanford.edu/news/all-news/2015/01/telomere-extension-turns-back-aging-clock-in-cultured-cells.html.
WHO. (2017 December 12). Mental health of older adults. World Health Organization. https://www.who.int/news-room/fact-sheets/detail/mental-health-of-older-adults.
WHO. (2018 February 5). Ageing and health. World Health Organization. https://www.who.int/news-room/fact-sheets/detail/ageing-and-health.
Yang, N., Sen, P. (2018). The senescent cell epigenome. Aging, 10(11), 3590-3609. https://doi.org/10.18632/aging.101617
Zhang, Y., Sun, X., Wang, Q., Xu, J., Dong, F., Yang, S., Yang, J., Zhang, Z., Qian, Y., Chen, J., Zhang, J., Liu, Y., Tao, R., Jiang, Y., Yang, J., Yang, S. (2020). Multicopy chromosomal integration using CRISPR-associated Transposases. ACS Synthetic Biology, 9(8), 1998-2008. https://doi.org/10.1021/acssynbio.0c00073

1-7. (canceled)

8. A method to extend telomeres in a genomic DNA strand comprising:
obtaining genomic DNA;
searching genomic DNA from for a telomere junction point joining a DNA proper and a telomere region;
forming a guide RNA comprising one or more telomere sequences and a sequence of said DNA proper adjacent to said telomere junction point;
contacting said guide RNA with said subject DNA and cleaving said subject DNA using a system guided by said guide RNA to form cleaved DNA proper;
contacting said cleaved DNA proper with a telomere extended DNA, said telomere extended DNA comprising at least said sequence of DNA proper from said guide RNA and a plurality of telomere sequences; and
joining said telomere extended DNA to said cleaved DNA proper.

9. A method of administering gene therapy to a subject comprising:
extracting a sample of genomic DNA from a subject;
replicating said genomic DNA to form replicated genomic DNA;
analyzing said replicated genomic DNA for the presence of an ideal or near-ideal genomic DNA sequence comprising a DNA proper sequence and a telomere sequence;
isolating said replicated genomic DNA having said idea or near-ideal genomic DNA sequence to form ideal genomic DNA;
replicating said ideal genomic DNA comprising a DNA proper and a telomere region;
forming a guide RNA comprising a sequence of said DNA proper and said telomere region; and
administering said guide RNA and a telomere extended DNA to said subject to replace at least a portion of subject DNA with said telomere extended DNA.

10. A method of searching genomic DNA of a subject comprising:
administering a guide RNA to a subject, said guide RNA comprising a sequence of an entire genomic DNA proper strand of said subject and a telomere region; or
administering a guide RNA to a subjects, said guide RNA comprising a sequence of an a portion of a genomic DNA proper strand of said subject and a telomere region.
